(19) United States
(12) Patent Application Publication
(10) Pub. No.: US 2026/0248849 A1
(43) Pub. Date: Aug. 27, 2026

(54) PROSTATE-SPECIFIC MEMBRANE ANTIGEN TARGETED NF-kB p50-DEFICIENT IMMATURE MYELOID CELLS

(71) Applicant: THE JOHNS HOPKINS UNIVERSITY, Baltimore, MD (US)

(72) Inventor: Alan D. Friedman, Baltimore, MD (US)

(73) Assignee: THE JOHNS HOPKINS UNIVERSITY, Baltimore, MD (US)

(21) Appl. No.: 19/664,318

(22) Filed: Apr. 30, 2026

Related U.S. Application Data
(63) Continuation of application No. PCT/US2024/053588, filed on Oct. 30, 2024.
(60) Provisional application No. 63/594,343, filed on Oct. 30, 2023.

(51) Int. Cl. A61K 35/15; A61K 40/10; A61K 40/31; A61K 40/42; A61P 35/00; C07K 14/705; C07K 16/30

(52) U.S. Cl. CPC A61K 35/15; A61K 40/10; A61K 40/31; A61K 40/4276; A61P 35/00; C07K 14/7051; C07K 14/70521; C07K 14/70578; C07K 16/3069; A61K 2239/13; A61K 2239/21; A61K 2239/22; A61K 2239/58; C07K 2317/54; C07K 2317/55; C07K 2317/565; C07K 2317/622

(57) ABSTRACT

The present disclosure relates to compositions comprising prostate-specific membrane antigen (PSMA) targeted p50-IMCs and methods of using such PSMA targeted p50-IMCs.

### CROSS-REFERENCE TO RELATED APPLICATIONS

This application is a continuation of International Application No. PCT/US2024/053558 filed Oct. 30, 2024, which claims priority to U.S. Provisional Application No. 63/594,343 filed Oct. 30, 2023, the contents of both of which are incorporated by reference it their entireties, and to which priority is claimed.

### GOVERNMENT SUPPORT CLAUSE

This invention was made with government support under grant W81XWH-21-1-0671 awarded by the US Army Military Medical Research and Development Command. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Oct. 30, 2024, is entitled “088933.0134_ST26.xml”, and is 7,723 bytes in size.

### FIELD

The present disclosure relates to compositions comprising prostate-specific membrane antigen (PSMA) targeted NF-κB p50-deficient immature myeloid cells (p50-IMCs) and methods of using such PSMA targeted p50-IMCs.

### BACKGROUND

Despite advances in both detection and treatment, prostate cancer remains a significant health issue worldwide. For example, while prostate cancer is second most diagnosed cancer, it continues to be the fifth leading cause of cancer-related death among men. (Wang et al., Front Pub. Health 20:81104 (2022)). Although new cell-based therapeutic modalities, e.g., PSMA-specific CAR-T cell therapies, are currently being investigated, questions remain as to their long-term efficacy and durability given art-recognized limitations to many cell-based therapeutics, e.g., the ability of tumor microenvironments to comprise T cell-inhibitory signals. Thus, there is a need in the art for therapeutic approaches that address such art-recognized limitations.

NF-κB p50-deficient immature myeloid cells (p50-IMCs), which exhibit absent or reduced expression of the NF-κB p50 protein subunit as compared to wild-type IMCs, have been shown to skew towards a proinflammatory phenotype, with increased cytokine expression and enhanced T cell activation. Such skewing occurs even in tumor microenvironments that would conventionally be associated suppression of antitumor T cell immunity and secretion of tumor-promoting growth factors. The adoptive transfer of p50-IMCs, for example, has been successful in slowing the growth of syngeneic murine prostate cancer when given after a dose of myelo-depleting 5-fluorouracil. Given such results, p50-IMCs may minimize tumor associated-inhibition of cell-based therapeutics.

In light of the significant health risks that remain associated with prostate cancer, and the relationship of p50-IMCs to cancer immunotherapy, additional therapeutic modalities involving p50-IMCs for treatment of prostate cancer are desirable.

### SUMMARY

The present disclosure relates to compositions comprising PSMA targeted p50-IMCs and methods of using such PSMA targeted p50-IMCs. In certain embodiments, the PSMA targeted p50-IMCs of the present disclosure comprise NF-κB p50-deficient immature myeloid cells comprising a PSMA targeting moiety. In certain embodiments, the PSMA targeting moiety comprises: (a) a surface bound anti-PSMA antibody; or (b) a surface expressed anti-PSMA chimeric antigen receptor (CAR).

In certain embodiments, the PSMA targeting moiety comprises: (a) a heavy chain variable region; and (b) a light chain variable region, wherein the heavy and light chain variable regions associate to bind PSMA. In certain embodiments, the PSMA targeting moiety comprises an scFv, wherein the scFv comprises a heavy chain variable region and a light chain variable region, wherein the heavy and light chain variable regions associate to bind PSMA. In certain embodiments, the extracellular antigen-binding domain comprises a heavy chain variable region comprising the heavy chain CDRs of Ab 3.9 or Ab 10.3 and a light chain variable region comprising the light chain CDRs of Ab 3.9 or 10.3.

In certain embodiments, the PSMA targeting moiety is a human scFv. In certain embodiments, the PSMA targeting moiety is a Fab, which is optionally crosslinked. In certain embodiments, the PSMA targeting moiety is a F (ab) 2. In certain embodiments, the PSMA targeting moiety comprises a linker between a heavy chain variable region and a light chain variable region of the PSMA targeting moiety.

In certain embodiments, the PSMA targeting moiety is a CAR comprising a transmembrane domain and an intracellular signaling domain. In certain embodiments, the transmembrane domain comprises a CD8 polypeptide, a CD28 polypeptide, a CD35 polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof. In certain embodiments, the intracellular signaling domain comprises a CD3 polypeptide. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof.

In certain embodiments, the PSMA targeting moiety is constitutively expressed on the surface of the cell.

In certain embodiments, the present disclosure is directed to a composition comprising the PSMA targeted p50-IMC described herein. In certain embodiments, the composition is a pharmaceutical composition comprising a pharmaceutically acceptable carrier.

In certain embodiments, the present disclosure is directed to methods of treating prostate cancer in a subject, comprising administering to the subject the presently disclosed PSMA targeted p50-IMC described herein. In certain embodiments, the subject is a human.

In certain embodiments, the present disclosure is directed to kits for treating prostate cancer in a subject, comprising the PSMA targeted p50-IMC disclosed herein. In certain embodiments, the kit further comprises written instructions for using the PSMA targeted p50-IMC or PSMA targeted p50-IMC composition for treating prostate cancer in a subject.

### BRIEF DESCRIPTION OF THE FIGURES

FIG. 1A-1C. p50: p50 represses myeloid cell NF-κB target genes in the basal state (1A). Pro-inflammatory cytokines induce NF-κB target gene activation (1B). NF-κB target gene de-repression in the absence of p50 (1C). RHD: Rel Homology Domain; TAD: Transactivation Domain.

FIG. 2A-2C. Exemplary PSMA targeted p50-IMCs can comprise a p50-IMC with surface-bound anti-PSMA antibody (2A, left) and/or p50-IMC expressing a PSMA CAR (2A, right). An exemplary PSMA CAR sequence illustration is provided (2B). The amino acid sequences of the leader, VH, linker, and VL domains in this PSMA.CAR are shown (2C).

FIG. 3A-3B. Purified anti-human PSMA Ab 3.9 (murine Ab) was obtained from the supernatant of a hybridoma line (ATCC PTA-3258). An anti-human PSMA.CAR cDNA was constructed utilizing the DNA sequences of the available variable regions of PSMA Ab 10.3 (VH, VL) to design a single-chain variable fragment (scFv), spacer and trans-membrane (TM) domains from human CD8, and an intra-cellular human CD3ζ domain, as diagrammed in FIG. 2B. Expression of PSMA.CAR was verified by transduction of Jurkat T cells utilizing an MIPuro-PSMA.CAR retroviral vector and flow cytometry using an anti-murine Fab Ab (3A, left). High affinity for PSMA was demonstrated by incubating these cells with PSMA-biotin followed by streptavidin (SA)-PerCP/Cy5.5 (3A, right). Human PSMA was introduced into murine MyC-CaP (MC) prostate cancer cells by retroviral transduction and puromycin selection, followed by flow-sorting of PSMA-high cells. As expression was low compared with PSMA in the human LNCaP cells, MC cells were developed expressing PSMA (NΔ9), lacking nine cytoplasmic residues required for spontaneous or Ab-induced PSMA cell internalization.

FIG. 4A-4C. Lineage-negative wild-type (WT) or p50−/−murine bone marrow cells were expanded, differentiated to macrophages, M1 or M2 polarized, incubated with PSMA antibody or isotype control, and mixed with CFSE-labeled MyC-CaP cells expressing human PSMA (NΔ9), as diagrammed (4A). Representative flow cytometry is presented (4B). Combined results of three experimental replicates are presented (4C).

FIG. 5A-5D. WT or p50−/−murine bone marrow cells were expanded, transduced with MIPuro or MIPuro-PSMA.CAR, puromycin-selected, differentiated to macrophages, M1 or M2 polarized, and mixed with CFSE-labeled MyC-CaP/PSMA (NΔ9) cells (5A). Expression of PSMA.CAR was confirmed by flow cytometry (5B). Representative flow cytometry are presented (5C). Results of three experimental replicates are presented (5D).

FIG. 6A-6D. 6A) Lineage-negative p50−/−murine bone marrow cells were expanded, cultured with M-CSF for one day to obtain p50-IMC, CFSE-labeled, incubated with PSMA antibody or IgG control, and injected into NSG mice bearing subcutaneous tumors derived from MyC/CaP-hPSMA (NΔ9) cells, +/−5FU five days prior to cell injection, as diagrammed (6A) Representative CD11b/CFSE flow cytometry is depicted in 6B. Tumor weight, total tumor CD11b+CSFE+ cells, and CD11b+CSFE+ cells per mg of tumor (mean, SE; n=4 per group). * p<0.05; ** p<0.01. Total tumor cells were enumerated using a hemocytometer after tumor dissociation multiplied by the percent of non-red blood cells based on flow gating (6C, 6D).

FIG. 7. Lineage-negative p50−/−murine bone marrow cells were expanded, cultured with M-CSF for one day to obtain p50-IMC, CFSE-labeled, incubated with PSMA antibody or IgG control, and injected into NSG mice bearing subcutaneous tumors derived from MyC/CaP-hPSMA (ND9) cells. 5FU was given five days prior to cell injection. Tumor weight, total tumor CD11b+CSFE+ cells, and CD11b+CSFE+ cells per mg of tumor (mean, SE). * p<0.05; ** p<0.01. Total tumor cells were enumerated using a hemocytometer after tumor dissociation and red blood cell lysis.

FIG. 8A-8B. 8A) Lineage-negative p50−/−murine bone marrow cells were expanded, transduced with vector or PSMA.CAR, cultured with M-CSF to obtain p50-IMC, CFSE-labeled, and injected into NSG mice bearing subcutaneous tumors derived from MyC/CaP-hPSMA (ND9) cells, as diagrammed. 8B) Tumor weight, total tumor CD11b+CSFE+ cells, and CD11b+CSFE+ cells per mg of tumor (mean, SE; n=3 per group).

FIG. 9A-9C. 9A) p50-IMCs were transduced with vector or PSMA.CAR, CFSE-labeled, and injected into NSG mice bearing tumors resulting from subcutaneous injection of prostate cells 28 days earlier. The mice received a dose of 5FU five days prior to p50-IMC injection. 9B) tumors were isolated, dissociated into single cells, and analyzed by flow cytometry. Representative flow cytometry for CD11b and CFSE is shown. 9C) mean tumor volumes, average total number of CD11b+CSFE+ cells that reached the tumors, and CD11b+CSFE+cells per mg of tumor weight is shown.

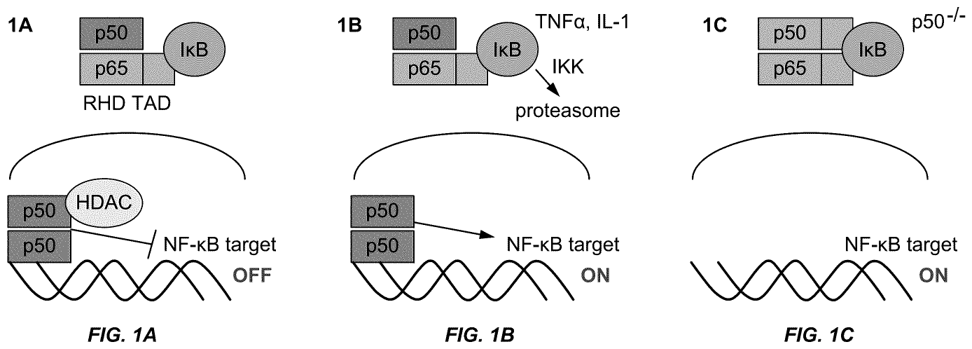

Leader sequence:
(SEQ ID NO.: 1)
MDWIWRILFLVGAATGAHS;
VH sequence:
(SEQ ID NO.: 2)
QVQLVESGGGVVQPGRSLRLSCAASGFAFSRYGMHWVRQAPGKGLEWVA
VIWYDGSNKYYADSVKGRFTISRDNSKNTQYLQMNSLRAEDTAVYYCAR
GGDFLYYYYYGMDVWGQGTTVTVSS;
linker:
(SEQ ID NO.: 3)
GGGGSGGGGSGGGGS;
VL sequence:
(SEQ ID NO.: 4)
DIQMTQSPSSLSASVGDRVTITCRASQGISNYLAWYQQKTGKVPKFLIY
QASTLQSGVPSRFSGGGSGTDFTLTISSLQPEDVATYYCQNYNSAPFTF
GPGTKVDIK;
assembled ocmponent sequence:
(SEQ ID NO.: 5)
MDWIWRILFLVGAATGAHSQVQLVESGGGVVQPGRSLRLSCAASGFAFS
RYGMHWVRQAPGKGLEWVAVIWYDGSNKYYADSVKGRFTISRDNSKNTQ
YLQMNSLRAEDTAVYYCARGGDFLYYYYYGMDVWGQGTTVTVSSGGGGS
GGGGSGGGGSDIQMTQSPSSLSASVGDRVTITCRASQGISNYLAWYQQK
TGKVPKFLIYQASTLQSGVPSRFSGGGSGTDFTLTISSLQPEDVATYYC
QNYNSAPFTFGPGTKVDIK.

### DETAILED DESCRIPTION

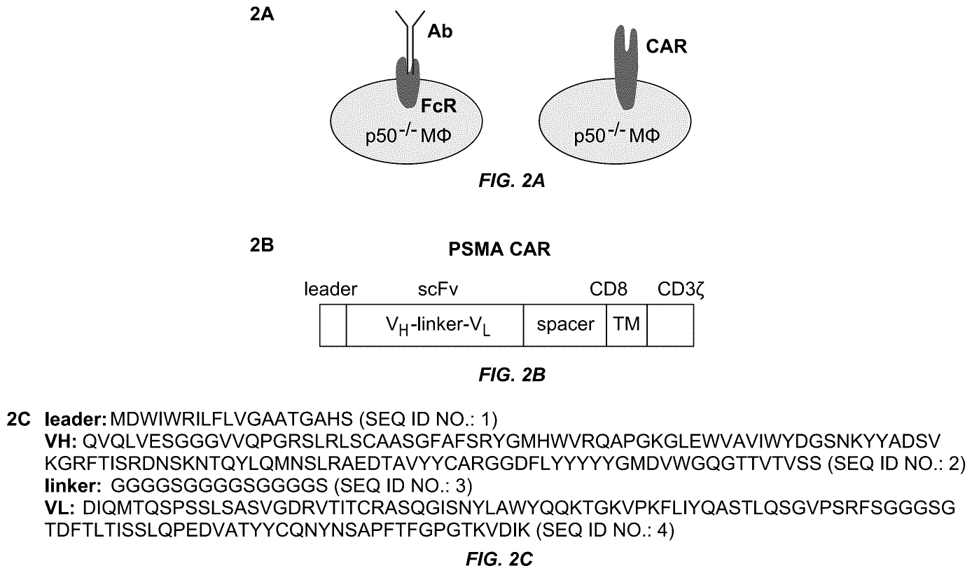

The present disclosure relates to compositions comprising PSMA targeted p50-IMCs and methods of using such PSMA targeted p50-IMCs. In certain embodiments, the PSMA targeted p50-IMCs of the present disclosure comprise p50-IMCs expressing a PSMA-specific CAR. For example, but not by way of limitation, the PSMA-specific CAR can comprise a target antigen binding domain, e.g., an scFv, that specifically binds PSMA. In certain embodiments, the PSMA targeted p50-IMCs of the present disclosure comprise p50-IMCs with surface-bound anti-PSMA antibody. Such exemplary PSMA targeted p50-IMCs are illustrated in FIG. 2A.

In certain embodiments, the PSMA targeted p50-IMCs of the present disclosure increase phagocytosis of tumor cells expressing PSMA. In certain embodiments, the PSMA targeted p50-IMCs of the present disclosure increase tumor localization of the p50-IMCs. In certain embodiments, the PSMA targeted p50-IMCs of the present disclosure can be used to treat cancer, e.g., prostate cancer, in a subject.

For clarity, but not by way of limitation, the detailed description of the presently disclosed subject matter is divided into the following subsections:
I. Definitions;
II. PSMA Targeted p50-IMC Compositions; and
III. Use of PSMA Targeted p50-IMC Compositions.

### I. Definitions

Unless defined otherwise, all technical and scientific terms used herein have the meaning commonly understood by a person skilled in the art to which the subject matter of the present disclosure belongs. The following references provide one of skill with a general definition of many of the terms used in the present disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the use of the word “a” or “an” when used in conjunction with the term “comprising” in the claims and/or the specification can mean “one,” but it is also consistent with the meaning of “one or more,” “at least one,” and “one or more than one.”

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “antibody” herein is used in the broadest sense and encompasses various antibody structures, including but not limited to monoclonal antibodies, polyclonal antibodies, multispecific antibodies (e.g., bispecific antibodies) and antibody fragments so long as they exhibit the desired antigen-binding activity.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2, diabodies, linear antibodies, single-chain antibody molecules (e.g., scFv) and multi-specific antibodies formed from antibody fragments.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat numbering system.

As used herein, the term “single-chain variable fragment” or “scFv” is a fusion protein of the variable regions of the heavy (VH) and light chains (VL) of an immunoglobulin (e.g., mouse or human) covalently linked to form a VH: VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80 (6): 1910-1917 (2008) and WO 2014/087010, the contents of which are hereby incorporated by reference in their entireties. In certain embodiments, the linker is a G4S linker.

The term “nucleic acid” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. The term nucleic acid encompasses deoxyribonucleic acid (DNA) including, e.g., complementary DNA (cDNA) and genomic DNA, ribonucleic acid (RNA), e.g., messenger RNA (mRNA), synthetic forms of DNA or RNA, and mixed polymers comprising two or more of these molecules. The nucleic acid molecule can be linear or circular. In addition, the term nucleic acid includes both, sense and antisense strands, as well as single stranded and double stranded forms. Moreover, the herein described nucleic acid can contain naturally occurring or non-naturally occurring nucleotides. Examples of non-naturally occurring nucleotides include modified nucleotide bases with derivatized sugars or phosphate backbone linkages or chemically modified residues.

The term “operative connection,” “operably linked” or “operatively linked,” as used herein, with regard to regulatory sequences of a nucleic acid, e.g., a gene, indicate an arrangement of elements in a combination enabling production of an appropriate effect. With respect to nucleic acids, e.g., genes, and regulatory sequences, an operative connection indicates a configuration of the nucleic acids, e.g., genes, with respect to the regulatory sequence allowing the regulatory sequences to directly or indirectly increase or decrease transcription or translation of the nucleic acids, e.g., genes. In particular, in certain embodiments, regulatory sequences directly increasing transcription of the operatively linked nucleic acid, e.g., gene, comprise promoters typically located on a same strand and upstream on a DNA sequence (towards the 5′ region of the sense strand), adjacent to the transcription start site of the nucleic acids, e.g., genes, whose transcription they initiate. In certain embodiments, regulatory sequences directly increasing transcription of the operatively linked nucleic acid, e.g., gene, comprise enhancers that can be located more distally from the transcription start site compared to promoters, and either upstream or downstream from the regulated nucleic acids, e.g., genes, as understood by those skilled in the art. Enhancers are typically short (50-1500 bp) regions of DNA that can be bound by transcriptional activators to increase transcription of a particular nucleic acid, e.g., gene. Typically, enhancers can be located up to 1 Mbp away from the nucleic acid, e.g., gene, upstream or downstream from the start site.

As used herein, “percentage of sequence identity” or “percentage of identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons. Sequence alignment algorithms also are disclosed in, for example, Altschul et al., J. Molecular Biol., 215 (3): 403-410 (1990); Beigert et al., Proc. Natl. Acad. Sci. USA, 106 (10): 3770-3775 (2009), Durbin et al., eds., Biological Sequence Analysis: Probalistic Models of Proteins and Nucleic Acids, Cambridge University Press, Cambridge, UK (2009); Soding, Bioinformatics, 21 (7): 951-960 (2005); Altschul et al., Nucleic Acids Res., (17): 3389-3402 (1997); and Gusfield, Algorithms on Strings, Trees and Sequences, Cambridge University Press, Cambridge UK (1997), each of which is incorporated herein by reference in its entirety).

The terms “comprise(s),” “include(s),” “having,” “has,” “can,” “contain(s)” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

As used herein, the term “individual” or “subject” refers to a vertebrate or an invertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, guinea pigs, rabbits, dogs, cats, sheep, pigs, goats, cattle, horses, apes and monkeys. In certain embodiments, the individual or subject is a human.

The term, “tumor” as used herein, means a neoplastic growth which may, or may not be malignant. Additionally, the compositions and methods provided herein are not only useful in the treatment of tumors, but in their micrometastases and their macrometastases. Typically, micrometastasis is a form of metastasis (the spread of a cancer from its original location to other sites in the body) in which the newly formed tumors are identified only by histologic examination; micrometastases are detectable by neither physical exam nor imaging techniques. In contrast, macrometastases are usually large secondary tumors.

The term “plurality” refers to a number larger than one. In certain embodiments, the term “plurality of cells” refers to a number of cells larger than one. For example, but not by way of limitation, a plurality of proteins includes at least two cells. In certain embodiments, the term “plurality of nucleic acids” refers to a number of nucleic acids larger than one. For example, but not by way of limitation, a plurality of nucleic acids includes at least two nucleic acids.

The term “specifically binds,” as used herein, refers to the preferential binding to a target molecule, e.g., a protein or nucleic acid, relative to other molecules, e.g., proteins or nucleic acids, in a sample.

As used herein, “treatment” (and grammatical variations thereof such as “treat” or “treating”) refers to clinical intervention in an attempt to alter the natural course of a disease in the individual being treated. Desirable effects of treatment include, but are not limited to alleviation of symptoms, diminishment of any direct or indirect pathological consequences of the disease, preventing metastasis, decreasing the rate of disease progression, amelioration or palliation of the disease state, and remission or improved prognosis. The decrease can be at least a 10%, 20%, 30%, 40%, 50%, 60%, 70%, 80%, 90%, 95%, 98% or 99% decrease in severity of complications, signs or symptoms or in likelihood of progression to another grade. “Treatment” can also refer to inhibiting proliferation of a cancer or progression to a higher grade by at least 10%, 20%, 30%, 40%, 50%, 60%, 70%, 80%, 90%, 95%, 98% or 99%.

### II. PSMA Targeted p50-IMC Compositions

In certain embodiments, the present disclosure relates to compositions comprising PSMA targeted p50-IMCs. In certain embodiments, the PSMA targeted p50-IMCs of the present disclosure can be generated from mammals lacking both copies of the gene encoding NF-κB p50. For example, but not by way of limitation, p50-IMCs can be obtained by expanding bone marrow cells from p50−/−mice using stem cell cytokines (SCF, FL, TPO), followed by transfer to M-CSF. Alternatively, the PSMA targeted p50-IMCs of the present disclosure can be generated from hematopoietic cells, such as those obtained from a cancer patient, using gene editing tools such as CRISPR/Cas9 to knockout one or both p50 gene alleles in a subset of the cells. In certain embodiments, the PSMA targeted p50-IMCs of the present disclosure are generated from hematopoietic cells, such as those obtained from a cancer patient, using agents that knockdown expression of p50 mRNA, such as shRNA, siRNA, anti-sense DNA, or anti-sense RNA.

In certain embodiments, the PSMA targeted p50-IMCs of the present disclosure express the monocyte markers CD11b, MCSFR, CD14, CD64, and/or CD16. In certain embodiments, the PSMA targeted p50-IMCs of the present disclosure express the dendritic cell markers HLA-DR, CD209, and/or FLT3. In certain embodiments, the PSMA targeted p50-IMCs of the present disclosure can also, or alternatively, express CD11c, CD1c, CD141, CD303, CD304, CD1a, CD15, CD13, and/or CD33.

In certain embodiments, the PSMA targeted p50-IMCs of the present disclosure comprise p50-IMCs further comprising a PSMA binding moiety. In certain embodiments, the PSMA targeted p50-IMCs of the present disclosure comprise p50-IMCs with surface-bound anti-PSMA antibody. For example, but not by way of limitation, such anti-PSMA antibodies can be bound to the surface of the p50-IMCs via an interaction between the Fc region of the anti-PSMA antibody and the Fc receptors present on the surface of the p50-IMCs. Alternative strategies for transiently or permanently binding anti-PSMA antibodies to the surface of the p50-IMCs are well-known in the art, e.g., modification of the anti-PSMA antibody to incorporate a p50-IMC surface binding moiety, conjugation of the anti-PSMA antibody to a lipid sufficient to induce p50-IMC surface binding via interaction with the p50-IMC cell membrane.

In certain embodiments, the PSMA targeted p50-IMCs of the present disclosure are prepared by contacting a plurality of p50-IMCs with a plurality of anti-PSMA antibodies. In certain embodiments, the plurality of anti-PSMA antibodies comprise a population of identical, or essentially identical, anti-PSMA antibodies (e.g., populations of identical antibodies may nevertheless exhibit some amino-terminal and/or carboxy-terminal amino acid heterogeneity). In certain embodiments, the plurality of anti-PSMA antibodies comprise a population of distinct anti-PSMA antibodies, e.g., the population of anti-PSMA antibodies can comprise sub-populations having one or more distinct CDR sequences relative to other sub-populations.

Numerous commercially available anti-PSMA antibodies known it the art that can be used in the context of the PSMA targeted p50-IMCs of the present disclosure. For example, but not by way limitation, exemplary anti-PSMA antibodies are disclosed in U.S. Pat. No. 7,850,971, which is incorporated by reference in its entirety. In certain embodiments the anti-PSMA antibody, e.g., Ab 3.9 and Ab 10.3, can be obtained from ATCC (PTA-3285 and PTA-3247). The VH and VL amino acid sequences of Ab 10.3 are shown in FIG. 2C. Because Ab 3.9 is fully murine it can be used in the context of immune-competent mice. Ab 10.3, on the other hand, is fully humanized and thus is appropriate for use with human p50-IMCs.

In certain embodiments, the PSMA binding moiety of the PSMA targeted p50-IMCs of the present disclosure is a PSMA-specific CAR. For example, but not by way of limitation, the PSMA-specific CAR can comprise a target antigen binding domain, e.g., an scFv, that specifically binds PSMA.

Generally, there are three generations of CARS. “First generation” CARs are typically composed of an extracellular antigen-binding domain (e.g., an scFv) fused to a transmembrane domain (e.g., a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof), which is fused to cytoplasmic/intracellular signaling domain. “First generation” CARs can provide activation of cells, including p50-IMC, through their CD33 chain signaling domain in a single fusion molecule. “Second generation” CARs add intracellular signaling domains from various co-stimulatory molecules (e.g., a CD28 polypeptide, a 4-1BB polypeptide, an OX40 polypeptide, an ICOS polypeptide, a DAP-10 polypeptide, or a combination thereof) to the cytoplasmic tail of the CAR to provide additional signals to the cell, including p50-IMC. “Second generation” CARs comprise those that provide both co-stimulation (e.g., CD28 or 4-1BB) and activation (CD32). “Third generation” CARs comprise those that provide multiple co-stimulation (e.g., CD28 and 4-1BB) and activation (CD3° C.). In certain embodiments, PSMA-specific CAR is a first-generation CAR. In certain embodiments, the PSMA-specific CAR is a second-generation CAR. In certain embodiments, the PSMA-specific CAR is a third generation CAR.

In certain embodiments, a cDNA encoding a PSMA-specific CAR (e.g., the “PSMA.CAR” described herein and illustrated in FIG. 2B) can be prepared using the available anti-PSMA Ab 10.3 variable region sequences. For example, but not by way of limitation a DNA sequence encoding a 19 amino acid leader peptide derived from human IgG (for membrane localization) can be synthesized in combination with an scFv domain comprising the Ab 10.3 VH and VL domains connected by a flexible linker. In certain embodiments, the resulting scFv can be fully humanized making it optimal for later clinical translation. To complete the CAR, the scFv encoding DNA can be ligated upstream of a spacer and a trans-membrane domain, e.g., a transmembrane domain derived from human CD8a, and an intracellular signaling domain, e.g., a signaling domain derived from hCD35. In certain embodiments, the hCD3ζ domain is employed as it has been reported to be sufficient to mediate phagocytosis by both murine and human macrophages. In certain embodiments, the cDNA encoding the PSMA.CAR can be introduced into a vector, e.g., a MIPuro retroviral vector, and the vector can be used to transduce p50-IMCs.

The present disclosure relates, in certain embodiments, to an NF-κB p50-deficient immature myeloid cell (p50-IMC) comprising a prostate-specific membrane antigen (PSMA) targeting moiety. In certain embodiments, the PSMA targeting moiety comprises: (a) a surface bound anti-PSMA antibody; or (b) a surface expressed anti-PSMA chimeric antigen receptor (CAR). In certain embodiments, the PSMA targeting moiety comprises a surface bound anti-PSMA antibody. In certain embodiments, the PSMA targeting moiety comprises a surface expressed anti-PSMA chimeric antigen receptor (CAR). In certain embodiments, the PSMA targeting moiety comprises: (a) a heavy chain variable region; and (b) a light chain variable region, wherein the heavy and light chain variable regions associate to bind PSMA. In certain embodiments the PSMA targeting moiety comprises an scFv, wherein the scFv comprises a heavy chain variable region and a light chain variable region, wherein the heavy and light chain variable regions associate to bind PSMA. In certain embodiments, the extracellular antigen-binding domain comprises a heavy chain variable region comprising the heavy chain CDRs of Ab 3.9 or Ab 10.3 and a light chain variable region comprising the light chain CDRs of Ab 3.9 or 10.3. In certain embodiments, the PSMA targeting moiety is a human scFv. In certain embodiments, the PSMA targeting moiety is a Fab, which is optionally crosslinked. In certain embodiments, the PSMA targeting moiety is a F (ab) 2. In certain embodiments, the PSMA targeting moiety comprises a linker between a heavy chain variable region and a light chain variable region of the PSMA targeting moiety. In certain embodiments, the PSMA targeting moiety is a CAR comprising a transmembrane domain and an intracellular signaling domain. In certain embodiments, the transmembrane domain comprises a CD8 polypeptide, a CD28 polypeptide, a CD32 polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof. In certain embodiments, the intracellular signaling domain comprises a CD32 polypeptide. In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the PSMA targeting moiety is constitutively expressed on the surface of the cell.

### III. Use of PSMA Targeted p50-IMC Compositions

The present disclosure relates to compositions comprising the PSMA targeted p50-IMC disclosed herein. In certain embodiments, the composition is a pharmaceutical composition, .e.g., where the composition further comprises a pharmaceutically acceptable carrier.

In certain embodiments, the PSMA targeted p50-IMCs of the present disclosure can be used to increase phagocytosis of tumor cells expressing PSMA. In certain embodiments, the PSMA targeted p50-IMCs of the present disclosure increase tumor localization of the p50-IMCs. In certain embodiments, the presently disclosed PSMA targeted p50-IMCs and compositions comprising thereof can be used for treating prostate cancer in a subject. In certain embodiments the subject is a human.

In certain embodiments, the PSMA targeted p50-IMC compositions of the present disclosure are pharmaceutical compositions comprising PSMA targeted p50-IMCs and a pharmaceutically acceptable carrier. Compositions comprising PSMA targeted p50-IMCs can be conveniently provided as sterile liquid preparations, e.g., isotonic aqueous solutions, which may be buffered to a selected pH. Liquid compositions can comprise carriers, for example, cell culture media with fetal bovine serum or serum-free cell culture media, phosphate-buffered or Hepes-buffered saline, and suitable mixtures thereof.

Sterile injectable solutions of the present disclosure can be prepared by incorporating the PSMA targeted p50-IMCs in the required amount of the appropriate media with various amounts of the other ingredients, as desired. Such PSMA targeted p50-IMC compositions may be in admixture with a suitable cell culture media, physiological saline, glucose, dextrose, or the like. The compositions can contain auxiliary substances such as pH buffering agents, antibiotics, and the like, depending upon the route of administration and the preparation desired.

Various additives which enhance the stability and sterility of the PSMA targeted p50-IMC compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The PSMA targeted p50-IMC compositions of the present disclosure can, in certain embodiments, be isotonic, i.e., they can have the same osmotic pressure as blood and lacrimal fluid. The desired isotonicity of the PSMA targeted p50-IMC compositions can be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the PSMA targeted p50-IMC compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected.

PSMA targeted p50-IMC compositions can be provided systemically or directly to a subject for treating or ameliorating prostate cancer. In certain embodiments, the PSMA targeted p50-IMC compositions comprising thereof are directly injected into an organ of interest (e.g., the prostate). Alternatively, the presently disclosed PSMA targeted p50-IMC compositions can be provided indirectly, for example, by administration into the circulatory system.

The quantity of PSMA targeted p50-IMCs to be administered can vary in view of the use, e.g., indication, and view of the subject being treated. In certain embodiments, between about 104 and about 10¹⁰, between about 104 and about 10⁷, between about 10⁵ and about 10⁷, between about 10⁵ and about 109, or between about 10⁶ and about 10⁸ of the PSMA targeted p50-IMCs are administered to a subject. More effective PSMA targeted p50-IMCs can be administered in even smaller numbers. Usually, at least about 1×10⁵ PSMA targeted p50-IMCs will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ PSMA targeted p50-IMCs are administered to a subject. In certain embodiments, about 1×10⁶ PSMA targeted p50-IMCs are administered to a subject. The precise determination of what would be considered an effective dose can be based on factors individual to each subject, including their size, age, sex, weight, and condition of the particular subject.

The PSMA targeted p50-IMCs of the present disclosure can, in certain embodiments, comprise a purified population of cells. Those skilled in the art can readily determine the percentage of PSMA targeted p50-IMCs in a population using various well-known methods, such as fluorescence activated cell sorting (FACS). Suitable ranges of purity in populations PSMA targeted p50-IMCs are about 50% to about 55%, about 55% to about 60%, 60% to about 65%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The PSMA targeted p50-IMCs can be introduced by injection, catheter, or the like.

The skilled artisan can readily determine the amount of PSMA targeted p50-IMCs and optional additives, vehicles, and/or carrier in compositions and to be administered in methods. Typically, any additives (in addition to the active cell(s) and/or agent(s)) are present in an amount of 0.001 to 20% of the volume of the solution. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein and, the time for sequential administrations can be ascertained without undue experimentation.

In certain embodiments, the PSMA targeted p50-IMCs of the present disclosure can be administered in combination with another therapeutic, e.g., chemotherapy. For example, but not by way of limitation, chemotherapy and the PSMA targeted p50-IMC cell transfer of the present disclosure can be performed sequentially or simultaneously. In certain embodiments, myeloid depleting chemotherapy may be conducted prior to adoptive cell transfer. The present disclosure is not limited by type of anti-cancer agent co-administered. Indeed, a variety of anti-cancer agents are contemplated to be useful in the present disclosure including, but not limited to, Acivicin; Aclarubicin; Acodazole Hydrochloride; Acronine; Adozelesin; Adriamycin; Aldesleukin; Alitretinoin; Allopurinol Sodium; Altretamine; Ambomycin; Ametantrone Acetate; Aminoglutethimide; Amsacrine; Anastrozole; Annonaceous Acetogenins; Anthramycin; Asimicin; Asparaginase; Asperlin; Azacitidine; Azetepa; Azotomycin; Batimastat; Benzodepa; Bexarotene; Bicalutamide; Bisantrene Hydrochloride; Bisnafide Dimesylate; Bizelesin; Bleomycin Sulfate; Brequinar Sodium; Bropirimine; Bullatacin; Busulfan; Cabergoline; Cactinomycin; Calusterone; Caracemide; Carbetimer; Carboplatin; Carmustine; Carubicin Hydrochloride; Carzelesin; Cedefingol; Celecoxib; Chlorambucil; Cirolemycin; Cisplatin; Cladribine; Crisnatol Mesylate; Cyclophosphamide; Cytarabine; Dacarbazine; DACA (N42-(Dimethyl-amino)ethyllacridine-4-carboxamide); Dactinomycin; Daunorubicin Hydrochloride; Daunomycin; Decitabine; Denileukin Diftitox; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; FK-317; FK-973; FR-66979; FR-900482; Gemcitabine; Geimcitabine Hydrochloride; Gemtuzumab Ozogamicin; Gold Au 198; Goserelin Acetate; Guanacone; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Methoxsalen; Metoprine; Meturedepa; Mitindomide; Mitocarcin; Mitocromin; Mitogillin; Mitomalcin; Mitomycin; Mytomycin C; Mitosper; Mitotane; Mitoxantrone Hydrochloride; Mycophenolic Acid; Nocodazole; Nogalamycin; Oprelvekin; Ormaplatin; Oxisuran; Paclitaxel; Pamidronate Disodium; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rituximab; Rogletimide; Rolliniastatin; Safingol; Safingol Hydrochloride; Samarium/Lexidronam; Semustine; Simtrazene; Sparfosate Sodium; Sparsomycin; Spirogermanium Hydrochloride; Spiromustine; Spiroplatin; Squamocin; Squamotacin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trastuzumab; Trestolone Acetate; Triciribine Phosphate; Trimetrexate; Trimetrexate Glucuronate; Triptorelin; Tubulozole Hydrochloride; Uracil Mustard; Uredepa; Valrubicin; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N,N′-Bis(2-chloroethyl)-N-nitrosourea (BCNU); N-(2-chloroethyl)-N′-cyclohex-yl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis(platinum); tomudex; azacitidine; cytarabine; gemcitabine; 6-Mercaptopurine; 6-Thioguanine; Hypoxanthine; teniposide; 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); and 2-chlorodeoxyadenosine (2-Cda).

It will be understood to those of skill in the art that the term “chemotherapeutic agent” is any agent capable of affecting the structure or function of the body of a subject or is an agent useful for the treatment or modulation of a disease or condition in a subject suffering therefrom. Examples of therapeutic agents can include any drugs known in the art for treatment of disease indications, including, for example, prostate cancer.

The dose of the chemotherapeutic agents used in conjunction with the PSMA targeted p50-IMCs of the present disclosure also will be determined by the existence, nature and extent of any adverse side effects that might accompany the administration of a particular composition. Typically, an attending physician will decide the dosage of the pharmaceutical composition with which to treat each individual subject, taking into consideration a variety of factors, such as age, body weight, general health, diet, sex, compound to be administered, route of administration, and the severity of the condition being treated. By way of example, and not intending to limit the invention, the dose of one or more chemotherapeutic agents used in conjunction with PSMA targeted p50-IMCs can be about 0.001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight.

In certain embodiments, the PSMA targeted p50-IMCs of the present disclosure can be administered in combination with another immunotherapy, e.g., an immune checkpoint inhibitor. For example, but not by way of limitation, PSMA targeted p50-IMC and additional immunotherapy can be performed sequentially or simultaneously. In certain embodiments, myeloid depleting chemotherapy may be conducted prior to p50-IMC therapy and an additional immunotherapy. The present application is not limited by type of anti-cancer immunotherapy administered co-administered. Indeed, a variety of anti-cancer immunotherapies are contemplated to be useful with p50-IMC in the present disclosure including, but not limited to, Atezolizumab, Avelumab, Durvalumab, Favezelimab, Ipilimumab, Nivolumab, and Pembrolizumab.

An active agent and a biologically active agent are used interchangeably herein to refer to a chemical or biological compound, including cells that induce a desired pharmacological and/or physiological effect, wherein the effect may be prophylactic or therapeutic.

The presently disclosed subject matter provides kits for treating prostate cancer in a subject. In certain embodiments, the kit comprises the presently disclosed PSMA targeted p50-IMCs or compositions comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the PSMA targeted p50-IMCs are provided together with instructions for administering the PSMA targeted p50-IMCs to a subject having or at risk of developing prostate cancer. The instructions generally include information about the use of the PSMA targeted p50-IMCs for the treatment and/or prevention of prostate cancer. In certain embodiments, the instructions include at least one of the following: description of the therapeutic agent; dosage schedule and administration for treatment or prevention of prostate cancer; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

The present disclosure relates, in certain embodiments, to methods of treating prostate cancer in a subject. In certain embodiments, such treatments will comprise administering to the subject a population of PSMA targeted p50-IMC or a composition comprising such cells as disclosed herein. In certain embodiments, the subject is a human.

The present disclosure also relates to kits for treating prostate cancer in a subject. In certain embodiments such kits will comprise a population of PSMA targeted p50-IMC or a composition comprising such cells as disclosed herein. In certain embodiments, the kit can further comprise written instructions for using the PSMA targeted p50-IMC or PSMA targeted p50-IMC composition for treating prostate cancer in a subject.

### EXAMPLES

The presently disclosed subject matter will be better understood by reference to the following example, which is provided as exemplary of the presently disclosed subject matter, and not by way of limitation.

### Example 1: Increased Phagocytosis of Cancer Cells via PSMA Targeting

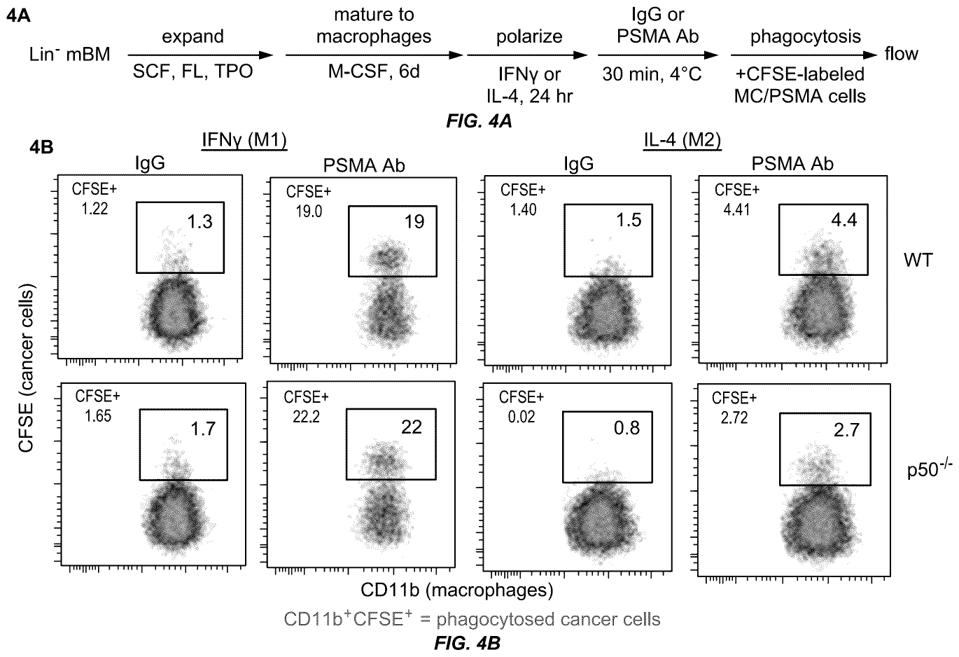

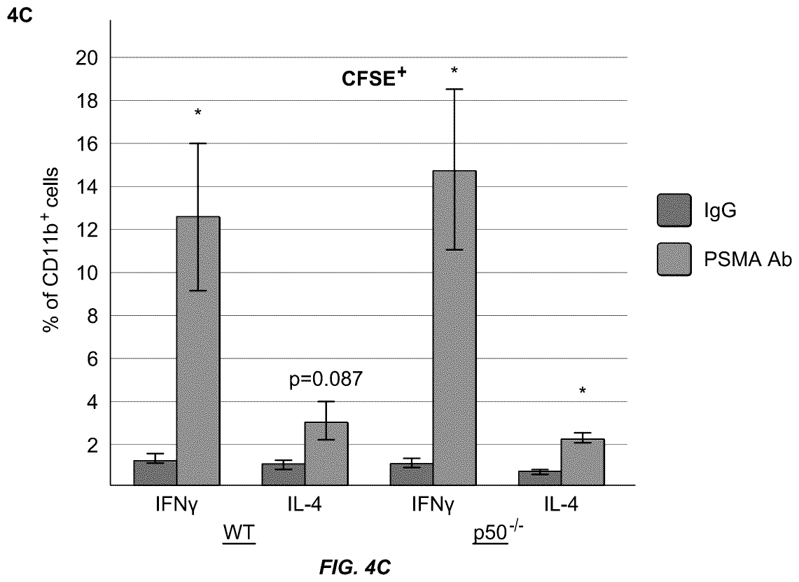

Combining p50-IMCs with an anti-PSMA antibody increases phagocytosis. The instant example is directed to determining whether combining p50-IMCs with an anti-PSMA antibody increases phagocytosis of prostate cancer cells by tumor macrophages developing from the p50-IMCs, expected to facilitate anti-tumor T cell activation via MHC antigen presentation. p50-IMCs, derived from the marrow of p50−/−mice, or WT-IMCs, derived from wild-type (WT), syngeneic B6 mice, were plated in M-CSF for six days to obtain bone marrow-derived macrophages (BMDM). These were then culture for 24 hours in either IFNγ or IL-4 to promote M1 or M2 gene expression, respectively. Of note, the prostate cancer tumor microenvironment favors the M2 macrophage phenotype. The macrophages were then released from the culture dishes and combined with either PSMA Ab3.9 or IgG1 isotype control Ab, each of which can bind to macrophages by interaction of the antibody Fc segment with the macrophage Fc Receptor. Ab- or IgG1-bound macrophages were then incubated with CFSE-dye labeled MC/PSMA (NΔ9) cells for 3 hours, followed by flow cytometry for CD11b (which detects the macrophages) and CFSE (which detect the prostate cancer cells), as diagrammed (FIG. 4A). Representative flow cytometry images are shown (FIG. 4B), and the results of three independent experiments are provided (FIG. 4C). Cells that are positive for both CD11b and CFSE represent macrophages that have phagocytosed the prostate cancer cells. To confirm that the macrophages have internalized the cancer cells, the macrophages can be labeled with the CFSE dye (green) and the MC/PSMA (NΔ9) cells with pHRodo Red dye, which is colorless at pH 7.5 (as in the culture media) but becomes red in the acidic environment of the phagocytic lysosome. Red cancer cells were only seen within green macrophages, confirming that phagocytosis has occurred. Cancer cells that have not been phagocytosed are colorless.

These results indicate that both WT and p50−/−macrophages have similar, low-level phagocytic activity in the absence of PSMA antibody. The presence of PSMA antibody, however, increases phagocytosis by both M1-polarized and M2-polarized WT or p50−/−macrophages. PSMA antibody-mediated phagocytosis is observed to be stronger with M1-polarized macrophages relative to M2-polarized macrophages.

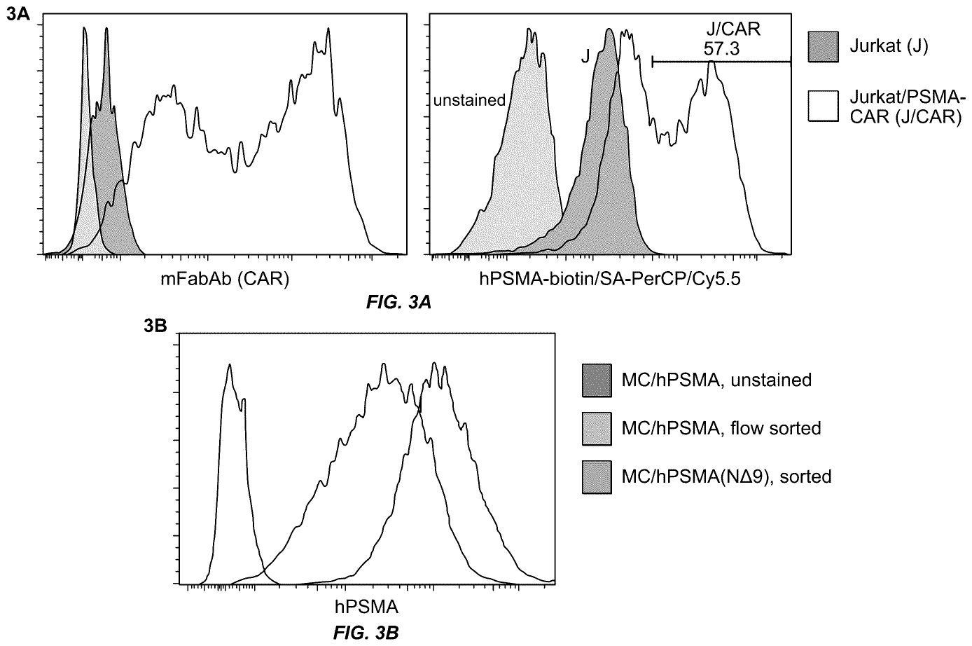

p50-IMCs expressing anti-PSMA CAR increases phagocytosis. The instant example is directed to determining whether p50-IMCs expressing an anti-PSMA CAR increase phagocytosis of prostate cancer cells. Expression of the PSMA.CAR construct (FIG. 2B) was verified by transduction of Jurkat T cells utilizing an MIPuro-PSMA.CAR retroviral vector and flow cytometry using an anti-murine Fab Ab (FIG. 3A, left). High affinity for PSMA was demonstrated by incubating these cells with PSMA-biotin followed by streptavidin (SA)-PerCP/Cy5.5 (FIG. 3A, right). Human PSMA was introduced into murine MyC-CaP (MC) prostate cancer cells by retroviral transduction and puromycin selection, followed by flow-sorting of PSMA-high cells. As expression was low compared with PSMA in the human LNCaP cells, MC cells were developed expressing PSMA (NΔ9), lacking nine cytoplasmic residues required for spontaneous or Ab-induced PSMA cell internalization (FIG. 3C).

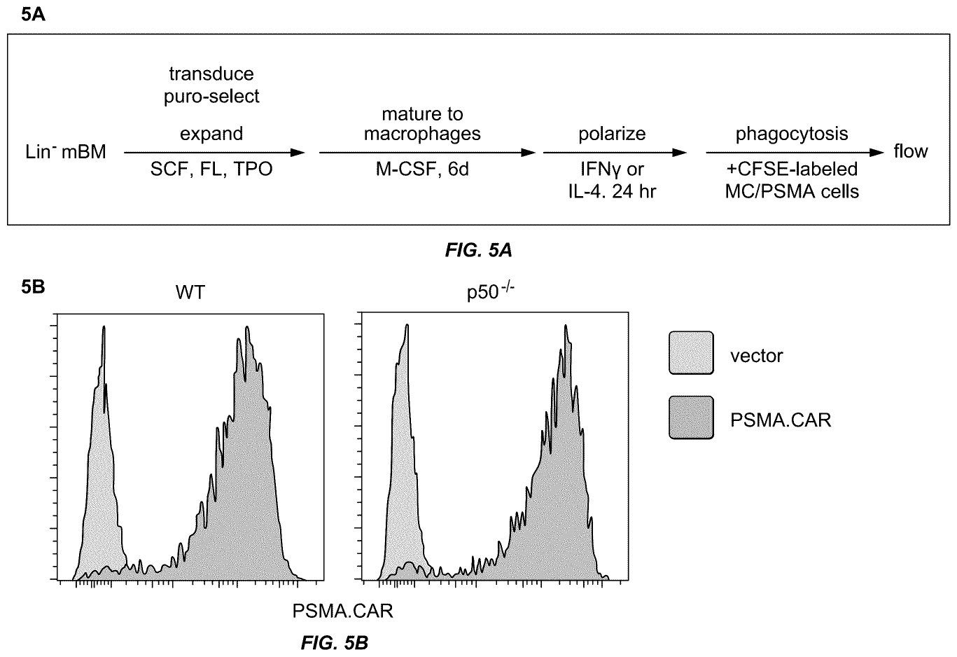

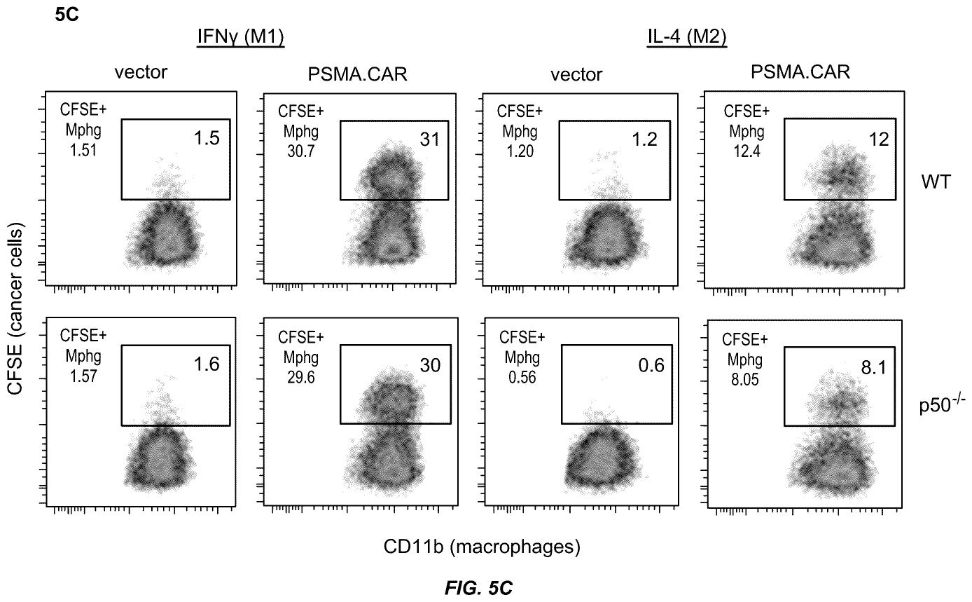

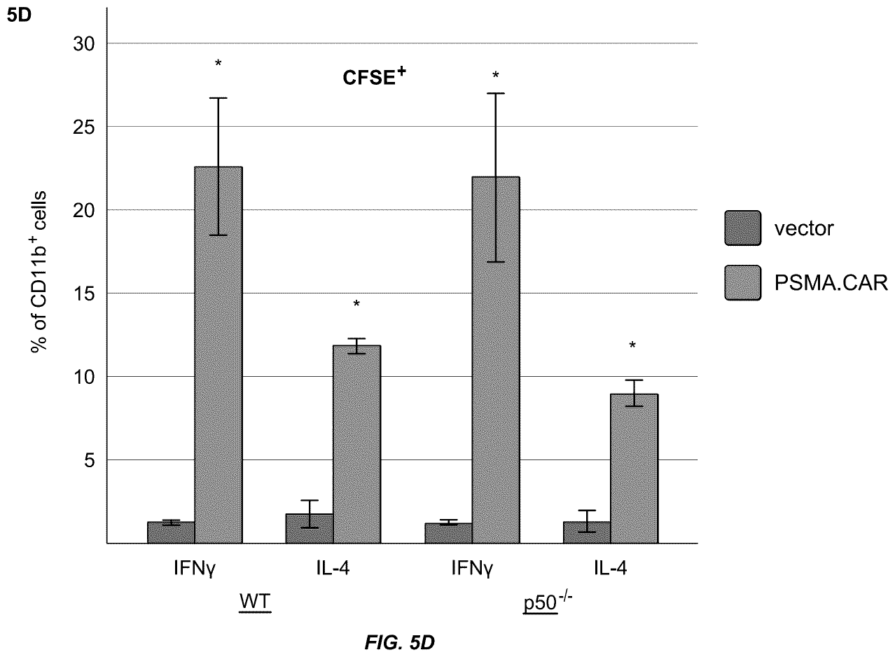

Macrophages developed from marrow cells transduced with PSMA.CAR or empty vector were incubated with CFSE-dye labeled MC/PSMA (NΔ9) cells for 3 hours, followed by flow cytometry for CD11b (which detects the macrophages) and CFSE (which detect the prostate cancer cells), as diagrammed (FIG. 5A). CAR expression in WT or p50−/−marrow cells after puromycin selection is shown (FIG. 5B). Representative flow cytometry images are shown (FIG. 5c), and the results of three independent experiments are provided (FIG. 5D). To confirm that the macrophages have internalized the cancer cells, the macrophages can be labelled with the CFSE dye (green) and the MC/PSMA (NΔ9) cells with pHRodo Red dye. Red cancer cells were only seen within green macrophages, confirming that phagocytosis has indeed occurred.

### Example 2: Increased Tumor Localization of p50-IMCs Via PSMA Targeting

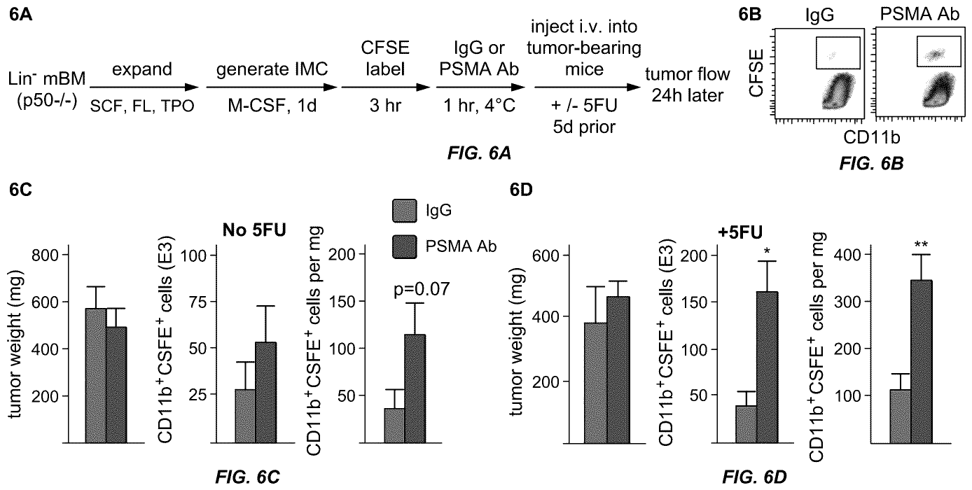

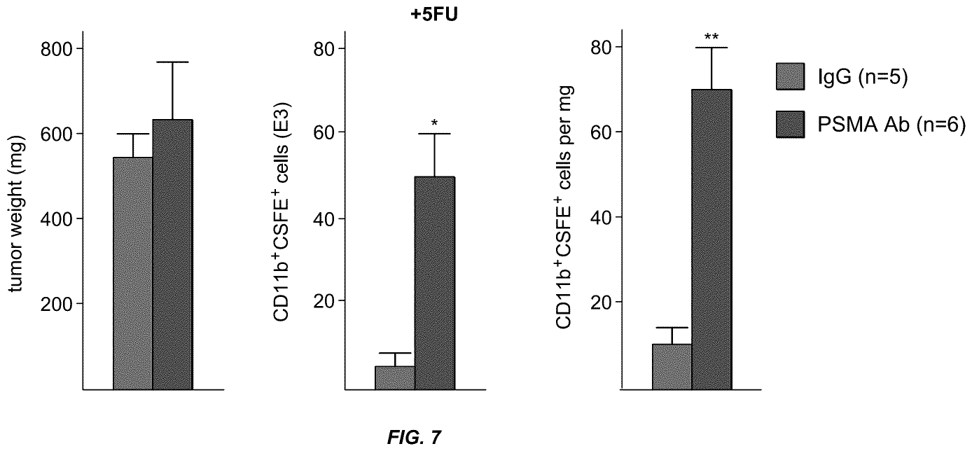

The instant example is directed to determining whether PSMA targeting increases p50-IMC localization to prostate tumor cells. To this end, p50-IMCs were CFSE-labeled, incubated with PSMA Ab3.9 or IgG, and injected into NSG mice bearing tumors resulting from subcutaneous injection of prostate cells 28 days earlier, followed by tumor isolation, dissociation into single cells and flow cytometry 24 hour later, as diagrammed (FIG. 6A). Representative tumor CD11b/CSFE flow cytometry shows increased tumor localization of p50-IMC bounds to PSMA Ab (FIG. 6B). In an initial experiment, the mice were not administered 5FU five days prior to intravenous p50-IMC injections. Mean tumor volumes, average total number of CD11b+CSFE+ cells that reached the tumors, and CD11b+CSFE+ cells per mg of tumor weight is shown (FIG. 6C, left). In a second experiment, 5FU was given five days prior to p50-IMC (FIG. 6C, right). In the absence of 5FU there was a trend towards increased tumor localization of labeled p50-IMCs when bound to PSMA antibody; in the presence of 5FU (which reduces circulating myeloid cells and tumor myeloid cells) PSMA antibody significantly increased the total number of labeled p50-IMCs and the number per mg of tumor weight that reached the prostate cancer tumors, compared with p50-IMCs bound to control IgG. Also, comparing No 5FU and +5FU, total CD11b+CSFE+ cells (40 vs 160 E3) and CD11b+CSFE+ cells/mg tumor (115 vs 340) are each significantly higher with 5FU, in the presence of PSMA antibody. When this experiment was repeated, with 5FU, total CD11b+CSFE+ cells (6 vs 50 E3) and CD11b+CSFE+ cells/mg tumor (10 vs 70) are again each significantly higher in the presence of PSMA antibody (FIG. 7).

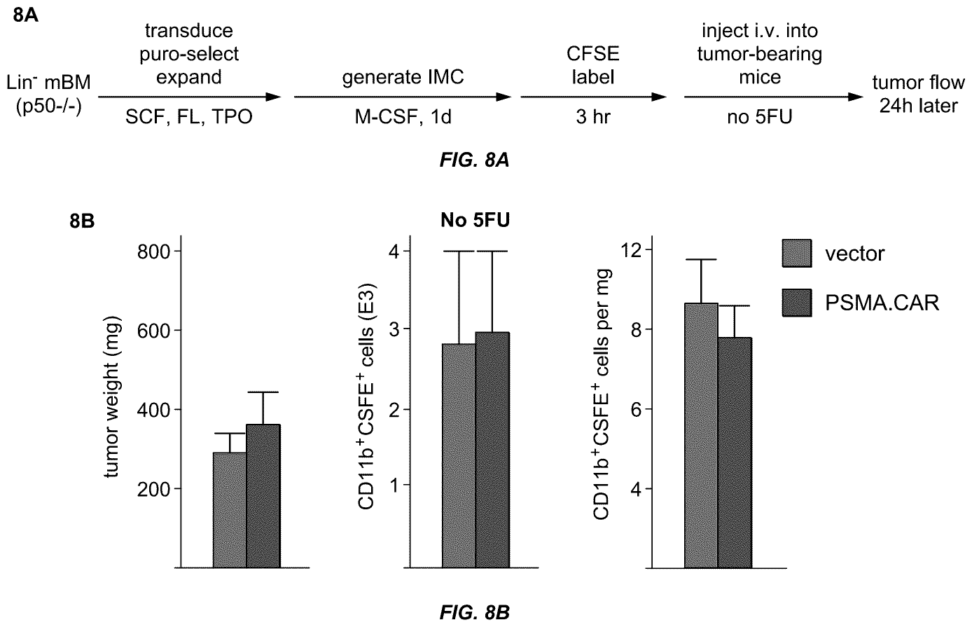

To evaluate whether PSMA.CAR expression increases p50-IMC localization to MC/PSMA (NΔ9) tumors in NSG mice, p50-IMCs transduced with vector or PSMA.CAR were CFSE-labeled and injected into NSG mice bearing tumors resulting from subcutaneous injection of prostate cells 28 days earlier, followed by tumor isolation, dissociation into single cells and flow cytometry 24 hour later, as diagrammed (FIG. 8A). In an initial experiment, no 5FU treatment five days prior to intravenous p50-IMC injections was administered. Mean tumor volumes, average total number of CD11b+CSFE+ cells that reached the tumors, and CD11b+CSFE+ cells per mg of tumor weight is shown (FIG. 8B). In this experiment, PSMA.CAR did not observably increase p50-IMC tumor localization.

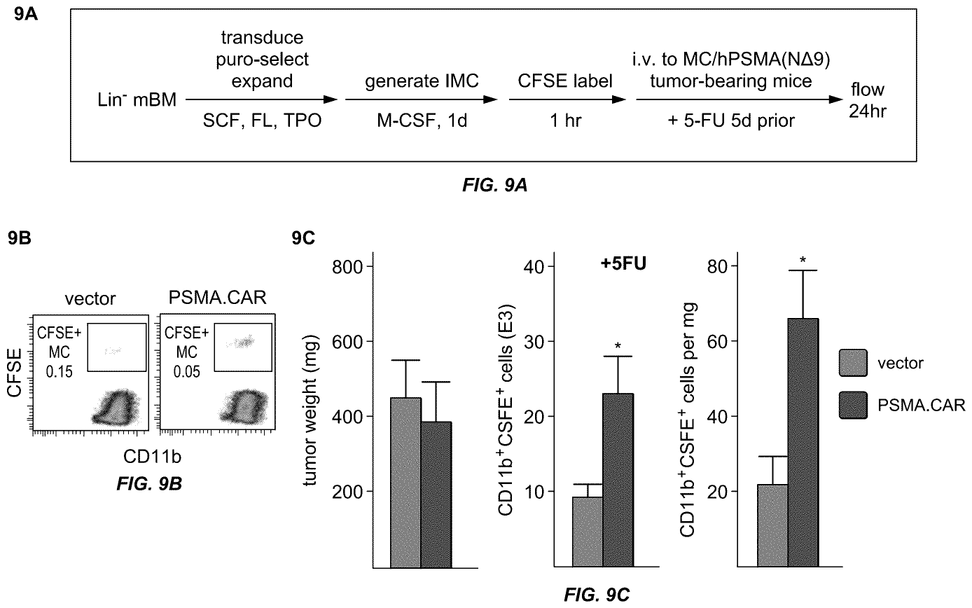

In a second experiment designed to test whether PSMA.CAR expression increases p50-IMC localization to MC/PSMA (NΔ9) tumors in NSG mice, p50-IMCs transduced with vector or PSMA.CAR were CFSE-labeled and injected into NSG mice bearing tumors resulting from subcutaneous injection of prostate cells 28 days earlier, followed by tumor isolation, dissociation into single cells and flow cytometry 24 hour later. In this experiment the mice received a dose of 5FU five days prior to p50-IMC injection, as diagrammed (FIG. 9A). Representative flow cytometry for CD11b and CFSE is shown (FIG. 9B). Mean tumor volumes, average total number of CD11b+CSFE+ cells that reached the tumors, and CD11b+CSFE+ cells per mg of tumor weight is shown (FIG. 9C). PSMA.CAR significantly increased p50-IMC tumor localization, by approximately 3-fold on average, as was also seen with PSMA antibody-bound p50-IMC, when mice received 5FU prior to p50-IMC injection.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. Accordingly, the appended claims are intended to comprise within their scope such processes, machines, manufacture, compositions of matter, means, methods or steps.

Various patents, patent applications, publications, product descriptions and protocols are cited throughout this application, the disclosure of which are incorporated herein by reference in their entireties for all purposes.

What is claimed is:

1. An NF-κB p50-deficient immature myeloid cell (p50-IMC) comprising a prostate-specific membrane antigen (PSMA) targeting moiety.

2. The PSMA targeted p50-IMC of claim 1, wherein PSMA targeting moiety comprises:
(a) a surface bound anti-PSMA antibody; or
(b) a surface expressed anti-PSMA chimeric antigen receptor (CAR).

3. The PSMA targeted p50-IMC of claim 1, wherein the PSMA targeting moiety comprises: (a) a heavy chain variable region; and (b) a light chain variable region, wherein the heavy and light chain variable regions associate to bind PSMA.

4. The PSMA targeted p50-IMC of claim 1, wherein the PSMA targeting moiety comprises an scFv, wherein the scFv comprises a heavy chain variable region and a light chain variable region, wherein the heavy and light chain variable regions associate to bind PSMA.

5. The PSMA targeted p50-IMC of claim 1, wherein the extracellular antigen-binding domain comprises a heavy chain variable region comprising the heavy chain CDRs of Ab 3.9 or Ab 10.3 and a light chain variable region comprising the light chain CDRs of Ab 3.9 or 10.3.

6. The PSMA targeted p50-IMC of claim 1, wherein the PSMA targeting moiety is a human scFv.

7. The PSMA targeted p50-IMC of claim 1, wherein the PSMA targeting moiety is a Fab, which is optionally crosslinked.

8. The PSMA targeted p50-IMC of claim 1, wherein the PSMA targeting moiety is a F (ab)₂.

9. The PSMA targeted p50-IMC of claim 1, wherein the PSMA targeting moiety comprises a linker between a heavy chain variable region and a light chain variable region of the PSMA targeting moiety.

10. The PSMA targeted p50-IMC of claim 1, wherein the PSMA targeting moiety is a CAR comprising a transmembrane domain and an intracellular signaling domain.

11. The PSMA targeted p50-IMC of claim 10, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

12. The PSMA targeted p50-IMC of claim 10, wherein the intracellular signaling domain comprises a CD3 polypeptide.

13. The PSMA targeted p50-IMC of claim 10, wherein the intracellular signaling domain further comprises at least one co-stimulatory signaling region.

14. The PSMA targeted p50-IMC of claim 13, wherein the at least one co-stimulatory signaling region comprises a CD28 polypeptide, a 4-1BB polypeptide, an OX40 polypeptide, an ICOS polypeptide, a DAP-10 polypeptide, or a combination thereof.

15. The PSMA targeted p50-IMC of claim 1, wherein the PSMA targeting moiety is constitutively expressed on the surface of the cell.

16. A method of treating prostate cancer in a subject, comprising administering to the subject the presently disclosed PSMA targeted p50-IMC of claim 1.

17. The method of claim 16, wherein the subject is a human.

18. A method of inducing phagocytosis of PSMA-expressing cells, comprising contacting such PSMA-expressing cells with the presently disclosed PSMA targeted p50-IMC of claim 1.

19. The method of claim 18, wherein said contacting comprises administering the PSMA targeted p50-IMC of claim 1.

20. The method of claim 19, wherein the subject is a human.


SEQUENCE LISTING (5 sequences)

SEQ ID NO: 1 - protein, 19 aa, synthetic construct
  REGION 1..19: note = Description of sequence: Construct component; type: leader
MDWIWRILFLVGAATGAHS

SEQ ID NO: 2 - protein, 123 aa, synthetic construct
  REGION 1..123: note = Description of sequence: Construct component; type: VH
QVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSS

SEQ ID NO: 3 - protein, 15 aa, synthetic construct
  REGION 1..15: note = Description of sequence: Construct component; type: linker
GGGGSGGGGSGGGGS

SEQ ID NO: 4 - protein, 107 aa, synthetic construct
  REGION 1..107: note = Description of sequence: Construct component; type: VL
DIQMTQSPSSLSASVGDRVTITCRASQGISNYLAWYQQKTGKVPKFLIYQASTLQSGVPSRFSGGGSGTDFTLTISSLQPEDVATYYCQNYNSAPFTFGPGTKVDIK

SEQ ID NO: 5 - protein, 264 aa, synthetic construct
  REGION 1..264: note = Description of sequence: Assembled components, leader, VH, linker and VL
MDWIWRILFLVGAATGAHSQVQLVESGGGVVQPGRSLRLSCAASGFAFSRYGMHWVRQAPGKGLEWVAVIWYDGSNKYYADSVKGRFTISRDNSKNTQYLQMNSLRAEDTAVYYCARGGDFLYYYYYGMDVWGQGTTVTVSSGGGGSGGGGSGGGGSDIQMTQSPSSLSASVGDRVTITCRASQGISNYLAWYQQKTGKVPKFLIYQASTLQSGVPSRFSGGGSGTDFTLTISSLQPEDVATYYCQNYNSAPFTFGPGTKVDIK